(19) United States
(12) Patent Application Publication
(10) Pub. No.: US 2008/0194868 A1
(43) Pub. Date: Aug. 14, 2008

(54) HYDRODYNAMIC CAVITATION CRYSTALLIZATION DEVICE AND PROCESS

(76) Inventor: Oleg V. Kozyuk, N. Ridgeville, OH (US)

Correspondence Address: BENESCH, FRIEDLANDER, COPLAN & ARONOFF LLP;ATTN: IP DEPARTMENT DOCKET CLERK, 2300 BP TOWER, 200 PUBLIC SQUARE, CLEVELAND, OH, 44114, US

(21) Appl. No.: 11/782,299

(22) Filed: Jul. 24, 2007

Related U.S. Application Data
(63) Continuation-in-part of application No. 11/330,473, filed on Jan. 12, 2006.
Application No. 11/330,473 is a division of application No. 10/382,117, filed on Mar. 4, 2003, now Pat. No. 7,041,144.

(51) Int. Cl. C01D 3/04; C07C 62/06

(52) U.S. Cl. 562/466; 23/303; 23/300

(57) ABSTRACT

A device and process for crystallizing a compound using hydrodynamic cavitation comprising the steps of mixing at least one stream of a solution of such compound to be crystallized with at least one stream of an anti-solvent and passing the mixed streams at an elevated pressure through a local constriction of flow to create hydrodynamic cavitation thereby causing nucleation and the direct production of crystals. The compound to be crystallized can be, for example, an active pharmaceutical ingredient.

### CROSS-REFERENCE TO RELATED APPLICATIONS

This application is a continuation-in-part of U.S. application Ser. No. 11/330,473 filed on Jan. 12, 2006, which is a divisional of U.S. application Ser. No. 10/382,117 filed on Mar. 4, 2003, which is now U.S. Pat. No. 7,041,144.

### BACKGROUND OF THE INVENTION

The present application relates to a device and process for crystallizing compounds using hydrodynamic cavitation.

The types of compounds that may be crystallized utilizing the devices and methods described herein include pharmaceutical compounds as well as any other compounds used in industry. Crystallization from solution of pharmaceutically active compounds or their intermediates is the typical method of purification used in industry. The integrity of the crystal structure, or crystal habit, that is produced and the particle size of the end product are important considerations in the crystallization process.

High bioavailability and short dissolution time are desirable or often necessary attributes of the pharmaceutical end product. However, the direct crystallization of small sized, high surface area particles is usually accomplished in a high supersaturation environment, which often results in material of low purity, high friability, and decreased stability due to poor crystal structure formation. Because the bonding forces in organic crystal lattices generate a much higher frequency of amorphism than those found in highly ionic inorganic solids, “oiling out” of supersaturated material is not uncommon, and such oils often solidify without structure.

Slow crystallization is a common technique used to increase product purity and produce a more stable crystal structure, but it is a process that decreases crystallizer productivity and produces large, low surface area particles that require subsequent high intensity milling. Currently, pharmaceutical compounds almost always require a post-crystallization milling step to increase particle surface area and thereby improve their bioavailability. However, high energy milling has drawbacks. Milling may result in yield loss, noise and dusting, as well as unwanted personnel exposure to highly potent pharmaceutical compounds. Also, stresses generated on crystal surfaces during milling can adversely affect labile compounds. Overall, the three most desirable end-product goals of high surface area, high chemical purity, and high stability cannot be optimized simultaneously using current crystallization technology without high energy milling.

One standard crystallization procedure involves contacting a supersaturated solution of the compound to be crystallized with an appropriate “anti-solvent” in a stirred vessel. Within the stirred vessel, the anti-solvent initiates primary nucleation which leads to crystal formation, sometimes with the help of seeding, and crystal digestion during an aging step. Mixing within the vessel can be achieved with a variety of agitators (e.g., Rushton or Pitched blade turbines, Intermig, etc.), and the process is done in a batchwise fashion.

When using current reverse addition technology for direct small particle crystallization, a concentration gradient can not be avoided during initial crystal formation because the introduction of feed solution to anti-solvent in the stirred vessel does not afford a thorough mixing of the two fluids prior to crystal formation. The existence of concentration gradients, and therefore a heterogeneous fluid environment at the point of initial crystal formation, impedes optimum crystal structure formation and increases impurity entrainment. If a slow crystallization technique is employed, more thorough mixing of the fluids can be attained prior to crystal formation which will improve crystal structure and purity, but the crystals produced will be large and milling will be necessary to meet bioavailability requirements.

Another standard crystallization procedure employs temperature variation of a solution of the material to be crystallized in order to bring the solution to its supersaturation point, but this is a slow process that produces large crystals. Also, despite the elimination of a solvent gradient with this procedure, the resulting crystal characteristics of size, purity and stability are difficult to control and are inconsistent from batch to batch.

Another crystallization procedure utilizes impinging jets to achieve high intensity micromixing in the crystallization process. High intensity micromixing is a well known technique where mixing-dependent reactions are involved. In U.S. Pat. No. 5,314,456 there is described a method using two impinging jets to achieve uniform particles. The general process involves two impinging liquid jets positioned within a well stirred flask to achieve high intensity micromixing. At the point where the two jets strike one another a very high level of supersaturation exists. As a result of this high supersaturation, crystallization occurs extremely rapidly within the small mixing volume at the impingement point of the two liquids. Since new crystals are constantly nuceleating at the impingement point, a very large number of crystals are produced. As a result of the large number of crystals formed, the average size remains small, although not all the crystals formed are small in size.

On the other hand, crystallization procedures using hydrodynamic cavitation have not yet been proposed. Cavitation is the formation of bubbles and cavities within a liquid stream resulting from a localized pressure drop in the liquid flow. If the pressure at some point decreases to a magnitude under which the liquid reaches the boiling point for this fluid, then a great number of vapor-filled cavities and bubbles are formed. As the pressure of the liquid then increases, vapor condensation takes place in the cavities and bubbles, and they collapse, creating very large pressure impulses and very high temperatures. According to some estimations, the temperature within the bubbles attains a magnitude on the order of 5000° C. and a pressure of approximately 500 kg/cm² (K. S. Suslick, Science, Vol. 247, 23 Mar. 1990, pgs. 1439-1445). Cavitation involves the entire sequence of events beginning with bubble formation through the collapse of the bubble. Because of this high energy level, it would be desirable to provide a device and process for crystallizing compounds using hydrodynamic cavitation. Devices and methods to create and control hydrodynamic cavitation are known in the art for use in mixing, conducting sonochemical type reactions, and preparing metal containing compounds, see e.g., U.S. Pat. Nos. 5,810,052, 5,931,771, 5,937,906, 6,012,492, and 6,365,555 to Kozyuk, which are hereby incorporated by reference in their entireties.

### BRIEF DESCRIPTION OF THE DRAWINGS

These and other features, aspects, and advantages of the present invention will become better understood with regard to the following description, appended claims, and accompanying drawings where:

FIG. 1 illustrates a longitudinal cross-section of one embodiment of a hydrodynamic cavitation crystallization device 10.

FIG. 2 illustrates a longitudinal cross-section of another embodiment of a hydrodynamic cavitation crystallization device 200.

FIG. 3 illustrates a longitudinal cross-section of another embodiment of a hydrodynamic cavitation crystallization device 300.

FIG. 4 illustrates a longitudinal cross-section of another embodiment of a hydrodynamic cavitation crystallization device 400.

FIG. 5 illustrates a longitudinal cross-section of another embodiment of a hydrodynamic cavitation crystallization device 500.

FIG. 6 illustrates a longitudinal cross-section of another embodiment of a hydrodynamic cavitation crystallization device 600.

FIG. 7 illustrates a longitudinal cross-section of another embodiment of a hydrodynamic cavitation crystallization device 700.

FIG. 8 illustrates a longitudinal cross-section of another embodiment of a hydrodynamic cavitation crystallization device 800.

FIG. 9 illustrates a longitudinal cross-section of another embodiment of a hydrodynamic cavitation crystallization device 900.

FIG. 10 illustrates a longitudinal cross-section of another embodiment of a hydrodynamic cavitation crystallization device 1000.

FIG. 11 illustrates a longitudinal cross-section of another embodiment of a hydrodynamic cavitation crystallization device 1100.

### DETAILED DESCRIPTION

In the description that follows, like parts are indicated throughout the specification and drawings with the same reference numerals, respectively. The figures are not drawn to scale and the proportions of certain parts have been exaggerated for convenience of illustration.

The present application describes devices and processes for crystallizing a compound using hydrodynamic cavitation. Compounds that can be crystallized utilizing these devices and methods include inorganic or organic materials. The organic material can include, for example, an active ingredient, such as an active pharmaceutical ingredient. Examples of active pharmaceutical ingredients include, without limitation, analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products, blood substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, parathyroid biphosphonates, prostaglandins, radio-pharmaceuticals, sex hormones, anti-allergic agents, stimulants, anoretics, sympathomimetics, thyroid agents, vasodilators, and xanthines.

Generally, the crystallization process begins with combining or mixing (e.g., by infusion) at least two fluid streams, at least two of which have different solvent compositions. For example, in a crystallization process that includes two fluid streams having different solvent compositions, one fluid is a solution of the compound to be crystallized in a suitable solvent or combination of solvents (the “feed solution”) and the other fluid is a suitable solvent or combination of solvents capable of initiating that compound's precipitation from solution (the “anti-solvent”). Preferably, the selected anti-solvent has a relatively low solvation property with respect to the crystalline compound. Examples of suitable solvents and anti-solvents include, without limitation, ethanol, methanol, ethyl acetate, halogenated solvents such as methylene chloride, acetonitrile, acetic acid, hexanes, ethers, and water.

Next, the crystallization process includes passing the combined or mixed fluid streams at an elevated pressure through a local constriction of flow to create hydrodynamic cavitation, thereby causing nucleation and the direct production of crystals. The size of the crystals is, for example, between about 0.01 microns and about 50 microns. Preferably, the crystal size can be between about 0.01 microns and about 5 microns. More preferably, the crystal size is between about 0.01 microns and about 1 micron.

Optionally, a surface modifier or a mixture of two or more surface modifiers can be added to the feed solution and/or the anti-solvent to alleviate agglomeration that might occur during the hydrodynamic cavitation crystallization process. The surface modifier(s) can be added as part of a premix or added through an introduction port in the device, which will be discussed in further detail below. It will be appreciated that since the surface modifier(s) may be incorporated in the crystalline compound, the surface modifier(s) should be one that is innocuous to the eventual use of the crystalline compound.

The surfaces modifiers that can be added to the feed solution and/or the anti-solvent include, without limitation, anionic surfactants, cationic surfactants, and nonionic surfactants. Examples of surface modifiers include, without limitation, gelatins, caseins, locithin, gum acacia, cholesterol, tragaeanth, stearic acid, benzalkonium chloride, calcium stearate, glyccryl monostearate, cetostearyl alcohol, cetomacrogol emulsifying wax, sorbitan esters, polyoxyethylcne alkyl ethers, polyoxyethylene caster oil derivatives, polyoxyethylene sorbitan htty acid esters, polyethylene glycols, polyoxyethylcne stearates, colloidel silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone, and phospholipids.

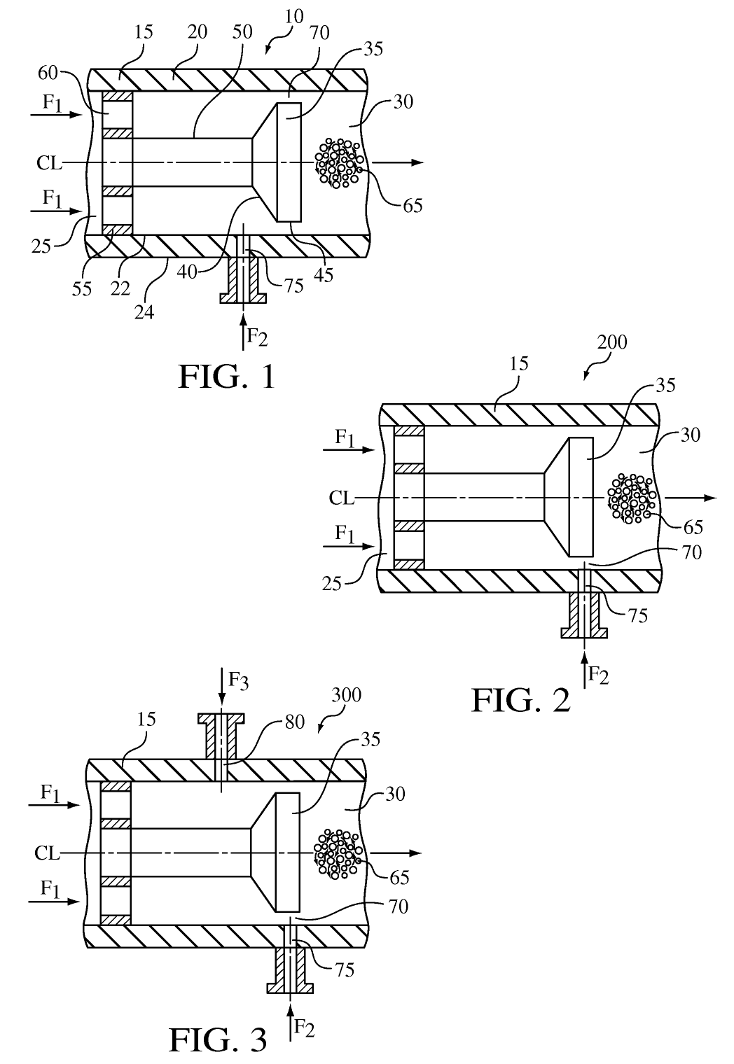

Referring now to the drawings, FIG. 1 illustrates a longitudal cross-section of one embodiment of a hydrodynamic cavitation crystallization device 10. The device 10 includes a flow-through channel 15 defined by a cylindrical wall 20 having an inner surface 22, an outer surface 24, an inlet 25 for introducing a first fluid stream F₁ (in the direction of the arrows) into device 10, and an outlet 30 for exiting fluid from the device 10. Although it is preferred that the cross-section of the flow-through channel 15 is circular, the cross-section of the flow-through channel 15 may take the form of any geometric shape such as square, rectangular, or hexagonal.

Disposed within the flow-through channel 15 along or near the centerline CL of the flow-through channel 15 is a cavitation generator such as a baffle 35. As shown in FIG. 1, the baffle 35 includes a conically-shaped surface 40 extending to a cylindrically-shaped surface 45 that confronts the fluid flow. The baffle 35 is positioned on a stem 50 that is connected to a disk 55 having orifices 60. The disk 55 is mounted in the inlet 25 and retains the baffle 35 inside the flow-through channel 15. In place of disk 55 having orifices 60, it is possible to use a crosshead, post, propeller or any other fixture that produces a minor loss of pressure.

The baffle 35 is configured to generate a hydrodynamic cavitation field 65 downstream via a local constriction 70 of fluid flow. In this embodiment, the local constriction 70 is an annular orifice defined between the inner surface 22 of the flow-through channel 15 and the cylindrically-shaped surface 45 of the baffle 35. Although the local constriction 70 is an annular orifice because of the cylindrically-shaped surface 45 of the baffle 35 and the circular cross-section of the cylindrical wall 20, it will be appreciated that if the cross-section of the flow-through channel 15 is any other geometric shape other than circular, then the local constriction 70 defined between the wall forming the flow-through channel 15 and the baffle 35 may not be annular in shape. Likewise, if the baffle 35 is not circular in cross-section, then the local constriction 70 defined between the wall forming the flow-through channel 15 and the baffle 35 may not be annular in shape. Preferably, the cross-sectional geometric shape of the wall forming the flow-through channel 15 matches the cross-sectional geometric shape of the baffle 35 (e.g., circular-circular, square-square, etc.).

To further promote the creation and control of cavitation fields downstream from the baffle 35, the baffle 35 can be constructed to be removable and replaceable by any baffle having a variety of shapes and configurations to generate varied hydrodynamic cavitation fields. The shape and configuration of the baffle 35 can significantly affect the character of the cavitation flow and, correspondingly, the quality of crystallization. Although there are an infinite variety of shapes and configurations that can be utilized within the scope of this invention, U.S. Pat. No. 5,969,207, issued on Oct. 19, 1999, discloses several acceptable baffle shapes and configurations, and U.S. Pat. No. 5,969,207 is hereby incorporated by reference in its entirety herein.

It will be appreciated that the baffle 35 can be removably mounted to the stem 50 in any acceptable fashion. However, it is preferred that the baffle 35 threadedly engages the stem 50. Therefore, in order to change the shape and configuration of the baffle 35, the stem 50 is removed from the device 10 and the original baffle 35 is unscrewed from the stem 50 and replaced by a different baffle element that is threadedly engaged to the stem 50 and replaced within the device 10.

Disposed in the cylindrical wall 20 of the flow-through channel 15 is a port 75 for introducing a second fluid stream F₂ (in the direction indicated by the arrow) into the flow-through channel 15. The port 75 is positioned in the cylindrical wall 20 of the flow-through channel 15 upstream from the baffle 35. In a slightly different embodiment as shown in FIG. 2, the device 200 includes a port 75 that is disposed in the cylindrical wall 20 of the flow-through channel 15 adjacent the local constriction 70 such that the second fluid stream F₂ mixes with the first fluid stream F₁ in the local constriction 70. In another embodiment as shown in FIG. 3, the device 300 includes a second port 80 disposed in the cylindrical wall 20 of the flow-through channel 15 to permit introduction of a third fluid stream F₃ (in the direction indicated by the arrow) into the flow-through channel 15. The second port 80 is positioned upstream from the baffle 35.

In operation of device 10 illustrated in FIG. 1, the first fluid stream F₁ enters the flow-through channel 15 via the inlet 25 and moves through the orifices 60 in the disk 55 in the direction represented by the arrows beneath F₁. The second fluid stream F₂ enters the flow-through channel 15 via the port 75 and mixes with the first fluid stream F₁ prior to confronting the baffle 35. In one embodiment, the first fluid stream F₁ is an anti-solvent and the second fluid stream F₂ is a feed solution. Alternatively, the first fluid stream F₁ is the feed solution and the second fluid stream F₂ is the anti-solvent. In the embodiment where the first fluid stream F₁ is the anti-solvent and the second fluid stream is the feed solution, the two fluid streams can be mixed by infusing the feed solution (i.e., the second fluid stream F₂) into the anti-solvent (i.e., the first fluid stream F₁).

The mixed first and second fluid streams F₁, F₂ then pass through the local constriction 70 of flow, where the velocity of the mixed first and second fluid streams F₁, F₂ increases to a minimum velocity (i.e., velocity at which cavitation bubbles begin to appear) dictated by the physical properties of the first and second fluid streams F₁, F₂. Optionally, instead of a single pass of the first fluid stream F₁ through the device 10, the first fluid stream F₁ (i.e., the anti-solvent) can be recirculated through the device 10, while the second fluid stream F₂ (i.e., the feed solution) is being introduced to the anti-solvent via the port 75. As the mixed first and second fluid streams F₁, F₂ pass through local constriction 70 of flow, a hydrodynamic cavitation field 65 (which generates cavitation bubbles) is formed downstream of the baffle 35. Upon reaching an elevated static pressure zone, the bubbles collapse causing high local pressures (to 5,000 kg/cm²) and temperatures (to 15,000° C.) to effect nucleation and thereby directly produce tiny crystals. The remaining fluids exit flow-through channel 15 via outlet 30, while the product crystals are isolated using conventional recovery techniques.

In operation of the device 200 illustrated in FIG. 2, the first fluid stream F₁ enters the flow-through channel 15 via the inlet 25 and moves through the orifices 60 in the disk 55 in the direction by the arrows beneath F₁. The second fluid stream F₂ enters the flow-through channel 15 via the port 75 and mixes with the first fluid stream F₁ while the first fluid stream F₁ is passing through the local constriction 70. In one embodiment, the first fluid stream F₁ is an anti-solvent and the second fluid stream F₂ is a feed solution. Alternatively, the first fluid stream F₁ is a feed solution and second fluid stream F₂ is an anti-solvent. In the embodiment where the first fluid stream F₁ is the anti-solvent and the second fluid stream is the feed solution, the two fluid streams can be mixed by infusing the feed solution (i.e., the second fluid stream F₂) into the anti-solvent (i.e., the first fluid stream F₁).

While passing through the local constriction 70 of flow, the velocity of the mixed first and second fluid streams F₁, F₂ increases to a minimum velocity (i.e., velocity at which cavitation bubbles begin to appear) dictated by the physical properties of the first and second fluid streams F₁, F₂. Optionally, instead of a single pass of the first fluid stream F₁ through the device 200, the first fluid stream F₁ (i.e., the anti-solvent) can be recirculated through the device 200, while the second fluid stream F₂ (i.e., the feed solution) is being introduced to the anti-solvent via the port 75. As the first and second fluid streams F₁, F₂ pass through the local constriction 70 of flow, the hydrodynamic cavitation field 65 (which generates cavitation bubbles) is formed downstream of the baffle 35. Upon reaching an elevated static pressure zone, the bubbles collapse causing high local pressures (to 5,000 kg/cm²) and temperatures (to 15,000° C.) to effect nucleation and thereby directly produce tiny crystals. The remaining fluids exit the flow-through channel 15 via the outlet 30, while the product crystals are isolated using conventional recovery techniques.

In operation of the device 300 illustrated in FIG. 3, the first fluid stream F₁ enters the flow-through channel 15 via the inlet 25 and moves through the orifices 60 in the disk 55 in the direction indicated by the arrows beneath F₁. The second the fluid stream F₂ enters the flow-through channel 15 via the second port 80 and mixes with the first fluid stream F₁ prior to confronting the baffle 35. The third fluid stream F₃ enters the flow-through channel 15 via the port 75 and mixes with the first and second fluid streams F₁, F₂ while they are passing through the local constriction 70. In one embodiment, the first fluid stream F₁ is an anti-solvent and the second and third fluid streams F₂, F₃ are the same or different feed solutions having the same or different concentrations. Alternatively, the first fluid stream F₁ is a feed solution, and the second and third fluid streams F₂, F₃ are the same or different anti-solvents having the same or different concentrations. In the embodiment where the first fluid stream F₁ is an anti-solvent and the second and third fluid streams F₂, F₃ are the same or different feed solutions having the same or different concentrations, the three fluid streams can be mixed by infusing the feed solution (i.e., the second and third fluid streams F₂, F₃) into the anti-solvent (i.e., the first fluid stream F₁).

While passing through the local constriction 70 of flow, the velocity of the mixed first, second, and third fluid streams F₁, F₂, F₃ increases to a minimum velocity (i.e., velocity at which cavitation bubbles begin to appear) dictated by the physical properties of the first, second, and third fluid streams F₁, F₂, F₃. Optionally, instead of a single pass of the first fluid stream F₁ through the device 300, the first fluid stream F₁ (i.e., the anti-solvent) can be recirculated through the device 300, while the second and third fluid streams F₂, F₃ (i.e., the feed solutions) are being introduced to the anti-solvent via the port 75 and the second port 80, respectively. As the first, second, and third fluid streams F₁, F₂, F₃ continue to pass through the local constriction 70 of flow, hydrodynamic cavitation field 65 (which generates cavitation bubbles) is formed downstream of the baffle 35. Upon reaching an elevated static pressure zone, the bubbles collapse causing high local pressures (to 5,000 kg/cm²) and temperatures (to 15,000° C.) to effect nucleation and thereby directly produce tiny crystals. The remaining fluids exit the flow-through channel 15 via the outlet 30, while the product crystals are isolated using conventional recovery techniques.

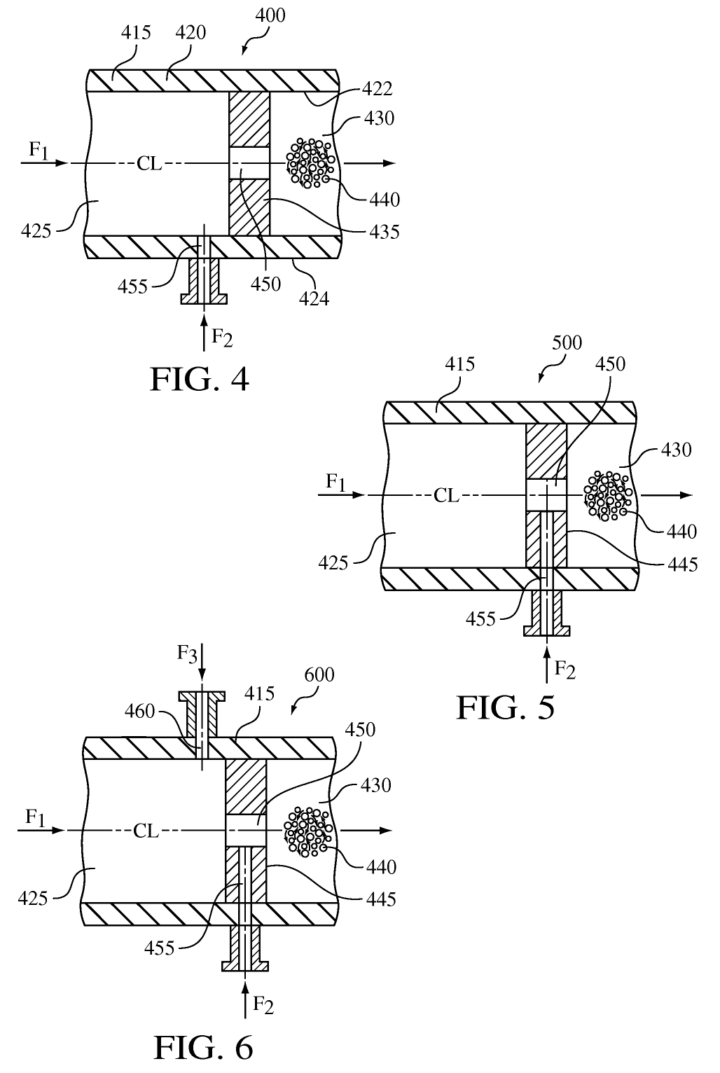

FIG. 4 illustrates another embodiment of a hydrodynamic cavitation crystallization device 400. The device 400 includes a flow-through channel 415 defined by a cylindrical wall 420 having an inner surface 422, an outer surface 424, an inlet 425 for introducing a first fluid stream F₁ (in the direction of the arrows) into the device 400, and an outlet 430 for exiting fluid from the device 400. Although it is preferred that the cross-section of the flow-through channel 415 is circular, the cross-section of the flow-through channel 415 may take the form of any geometric shape such as square, rectangular, or hexagonal and still be within the scope of the present invention.

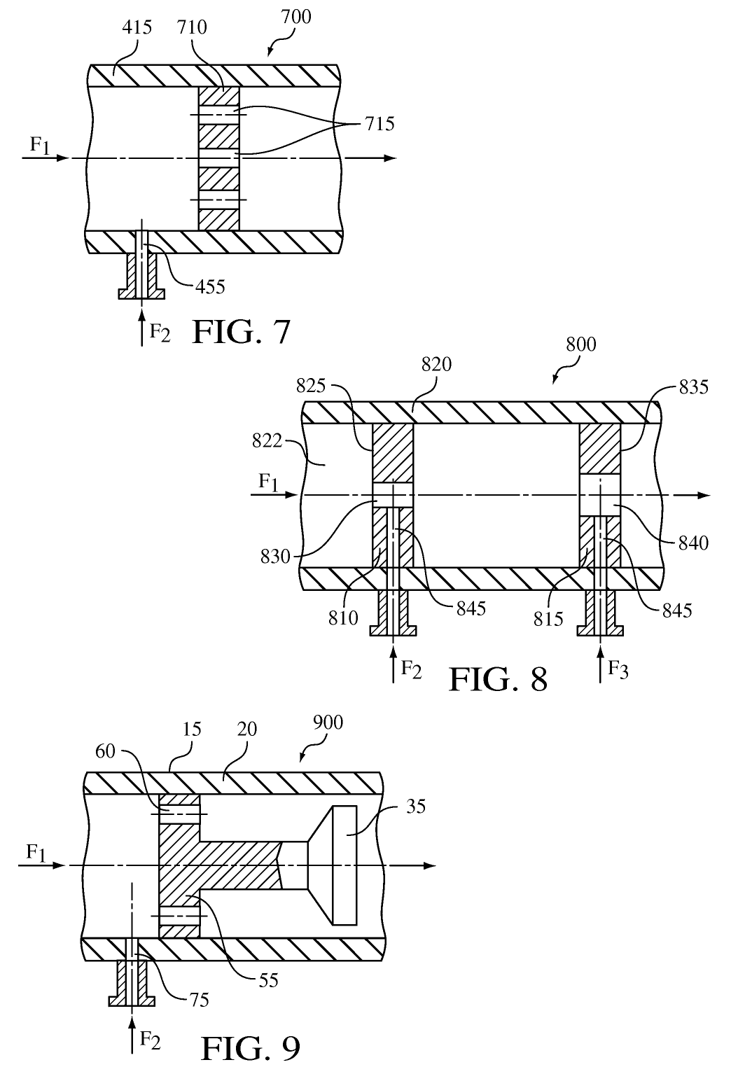

Disposed within the flow-through channel 415 is a cavitation generator 435 configured to generate a hydrodynamic cavitation field 440 downstream from the cavitation generator 435. As shown in FIG. 4, the cavitation generator 435 is a disk 445 having a circular orifice 450 disposed therein situated along or near the centerline CL of the flow-through channel 415. The orifice 450 is in the shape of Venturi tube and produces a local constriction of fluid flow. In a slightly different embodiment as shown in FIG. 7, the device 700 includes a disk 710 having multiple circular orifices 715 disposed therein to produce multiple local constrictions of fluid flow. Although it is preferred that the cross-section of the orifices in the disc are circular, the cross-section of the orifice may take the form of any geometric shape such as square, rectangular, or hexagonal and still be within the scope of the present invention.

To further promote the creation and control of the cavitation fields downstream from the disk 445 having an orifice 450, the disk 445 having an orifice 450 is constructed to be removable and replaceable by any disk having an orifice shaped and configured in a variety of ways to generate varied hydrodynamic cavitation fields. The shape and configuration of the orifice 450 can significantly affect the character of the cavitation flow and, correspondingly, the quality of crystallization. Although there are an infinite variety of shapes and configurations that can be utilized within the scope of this invention, U.S. Pat. No. 5,969,207, issued on Oct. 19, 1999, discloses several acceptable baffle shapes and configurations, and U.S. Pat. No. 5,969,207 is hereby incorporated by reference in its entirety herein.

Disposed in the cylindrical wall 420 of the flow-through channel 415 is an entry port 455 for introducing a second fluid stream F₂ (in the direction of the arrows) into the flow-through channel 415. The port 455 is disposed in the cylindrical wall 420 of the flow-through channel 415 upstream from the disk 445. In a slightly different embodiment as shown in FIG. 5, the device 500 includes a port 455 disposed in the cylindrical wall 420 of the flow-through channel 415 and extending through the disk 445 such that the port 455 is in fluid communication with the orifice 450. Thus, the second fluid stream F₂ mixes with the first fluid stream F₁ in the orifice 450. In yet another embodiment as shown in FIG. 6, the device 600 includes a second port 460 disposed in cylindrical wall 420 of flow-through channel 415 to permit introduction of a third fluid stream F₃ into flow-through channel 415. The second port 460 is positioned upstream from the disk 445.

In operation of the device 400 illustrated in FIG. 4, the first fluid stream F₁ enters the flow-through channel 415 via the inlet 425 and moves through the flow-through channel 415 along the direction indicated by the arrow beneath F₁. The second fluid stream F₂ enters the flow-through channel 415 via the entry port 455 and mixes with the first fluid stream F₁ prior to passing through the orifice 450. In one embodiment, the first fluid stream F₁ is an anti-solvent and the second fluid stream F₂ is a feed solution. Alternatively, the first fluid stream F₁ is a feed solution and second fluid stream F₂ is an anti-solvent. In the embodiment where the first fluid stream F₁ is the anti-solvent and the second fluid stream is the feed solution, the two fluid streams can be mixed by infusing the feed solution (i.e., the second fluid stream F₂) into the anti-solvent (i.e., the first fluid stream F₁).

The mixed first and second fluid streams F₁, F₂ then pass through the orifice 450, where the velocity of the first and second fluid streams F₁, F₂ increases to a minimum velocity (i.e., velocity at which cavitation bubbles begin to appear) dictated by the physical properties of the first and second fluid streams F₁, F₂. Optionally, instead of a single pass of the first fluid stream F₁ through the device 400, the first fluid stream F₁ (i.e., the anti-solvent) can be recirculated through the device 400, while the second fluid stream F₂ (i.e., the feed solution) is being introduced to the anti-solvent via the port 455. As the first and second fluid streams F₁, F₂ pass through the orifice 450, the hydrodynamic cavitation field 440 (which generates cavitation bubbles) is formed downstream of the orifice 450. Upon reaching an elevated static pressure zone, the bubbles collapse causing high local pressures (to 5,000 kg/cm²) and temperatures (to 15,000° C.) to effect nucleation and thereby directly produce tiny crystals. The remaining fluids exit the flow-through channel 415 via the outlet 430, while the product crystals are isolated using conventional recovery techniques.

In operation of the device 500 illustrated in FIG. 5, the first fluid stream F₁ enters the flow-through channel 415 via the inlet 425 and moves through the flow-through channel 415 along the direction indicated by the arrow beneath F₁. The second fluid stream F₂ enters the flow-through channel 415 via the entry port 455 and mixes with the first fluid stream F₁ while the first fluid stream F₁ is passing through the orifice 450. In one embodiment, the first fluid stream F₁ is an anti-solvent and the second fluid stream F₂ is a feed solution. Alternatively, the first fluid stream F₁ is a feed solution and the second fluid stream F₂ is an anti-solvent. In the embodiment where the first fluid stream F₁ is the anti-solvent and the second fluid stream is the feed solution, the two fluid streams can be mixed by infusing the feed solution (i.e., the second fluid stream F₂) into the anti-solvent (i.e., the first fluid stream F₁).

While passing through the orifice 450, the velocity of mixed first and second fluid streams F₁, F₂ increases to a minimum velocity (i.e., velocity at which cavitation bubbles begin to appear) dictated by the physical properties of the first and second fluid streams F₁, F₂. Optionally, instead of a single pass of the first fluid stream F₁ through the device 500, the first fluid stream F₁ (i.e., the anti-solvent) can be recirculated through the device 500, while the second fluid stream F₂ (i.e., the feed solution) is being introduced to the anti-solvent via the port 455. As the first and second fluid streams F₁, F₂ pass through the orifice 450, the hydrodynamic cavitation field 440 (which generates cavitation bubbles) is formed downstream of the orifice 450. Upon reaching an elevated static pressure zone, the bubbles collapse causing high local pressures (to 5,000 kg/cm²) and temperatures (to 15,000° C.) to effect nucleation and thereby directly produce tiny crystals. The remaining fluids exit the flow-through channel 415 via the outlet 430, while the product crystals are isolated using conventional recovery techniques.

In operation of the device 600 illustrated in FIG. 6, the first fluid stream F₁ enters the flow-through channel 415 via the inlet 425 and moves through the flow-through channel 415 along the direction indicated by the arrow beneath F₁. The second fluid stream F₂ enters the flow-through channel 415 via the second port 460 and mixes with the first fluid stream F₁ prior to passing through the orifice 450. The third fluid stream F₃ enters the flow-through channel 415 via the entry port 455 and mixes with the first and second fluid streams F₁, F₂ while they are passing through the orifice 450. In one embodiment, the first fluid stream F₁ is an anti-solvent and the second and third fluid streams F₂, F₃ are the same or different feed solutions having the same or different concentrations. Alternatively, in another embodiment, the first fluid stream F₁ is a feed solution, and the second and third fluid streams F₂, F₃ are the same or different anti-solvents having the same or different concentrations. In the embodiment where the first fluid stream F₁ is an anti-solvent and the second and third fluid streams F₂, F₃ are the same or different feed solutions having the same or different concentrations, the three fluid streams can be mixed by infusing the feed solution (i.e., the second and third fluid streams F₂, F₃) into the anti-solvent (i.e., the first fluid stream F₁).

While passing through the orifice 450, the velocity of mixed first, second, and third fluid streams F₁, F₂, F₃ increases to a minimum velocity (i.e., velocity at which cavitation bubbles begin to appear) dictated by the physical properties of the first, second, and third fluid streams F₁, F₂, F₃. Optionally, instead of a single pass of the first fluid stream F₁ through the device 600, the first fluid stream F₁ (i.e., the anti-solvent) can be recirculated through the device 600, while the second and third fluid streams F₂, F₃ (i.e., the feed solutions) are being introduced to the anti-solvent via the port 455 and the second port 460, respectively. As the first, second, and third fluid streams F₁, F₂, F₃ continue to pass through the orifice 450, the hydrodynamic cavitation field 440 (which generates cavitation bubbles) is formed downstream of the orifice 450. Upon reaching an elevated static pressure zone, the bubbles collapse causing high local pressures (to 5,000 kg/cm²) and temperatures (to 15,000° C.) to effect nucleation and thereby directly produce tiny crystals. The remaining fluids exit the flow-through channel 415 via the outlet 430, while the product crystals isolated using conventional recovery techniques.

FIG. 8 illustrates another embodiment of a hydrodynamic cavitation crystallization device 800, which is similar to the device 500 illustrated in FIG. 5 in structure and operation, except that the device 800 includes two cavitation generators 810, 815 arranged in series in the flow-through channel 820 to create two stages of hydrodynamic cavitation. The flow-through channel 820 includes an inlet 822 to introduce a first fluid stream F₁ (in the direction of the arrows). The first cavitation generator 810 is a disk 825 positioned within the flow-through channel 820 and includes a first orifice 830 disposed therein having a diameter. The second cavitation generator 815 is a disk 835 positioned within the flow-through channel 820 and includes a second orifice 840 having a diameter that is greater than the first diameter of the first orifice 830. In another embodiment, the diameter of the first orifice 830 may be greater than the diameter of the second orifice 840.

Disposed in the wall of the flow-through channel 820 and in fluid communication with the first orifice 830 and the second orifice 840 are the first port 845 and the second port 850, respectively, for introducing a second fluid stream F₂ and a third fluid stream F₃. In one embodiment, the first fluid stream F₁ is an anti-solvent and the second and third fluid streams F₂, F₃ are the same or different feed solutions having the same or different concentrations. Alternatively, the first fluid stream F₁ is a feed solution, and the second and third fluid streams F₂, F₃ are the same or different anti-solvents having the same or different concentrations. In the embodiment where the first fluid stream F₁ is an anti-solvent and the second and third fluid streams F₂, F₃ are the same or different feed solutions having the same or different concentrations, the three fluid streams can be mixed by infusing the feed solution (i.e., the second and third fluid streams F₂, F₃) into the anti-solvent (i.e., the first fluid stream F₁).

FIG. 9 illustrates another embodiment of a hydrodynamic cavitation crystallization device 900, which is similar to the device 100 illustrated in FIG. 1 in structure and operation, except that the port 75 is disposed in cylindrical wall 20 of the flow-through channel 15 and positioned in the cylindrical wall 20 of the flow-through channel 15 upstream from the disk 55. By positioning the port 75 upstream from the disk 55, the device 900 essentially creates two stages of hydrodynamic cavitation. In other words, the disk 55 having orifices 60 is the first stage of cavitation and the baffle 35 is the second stage of cavitation.

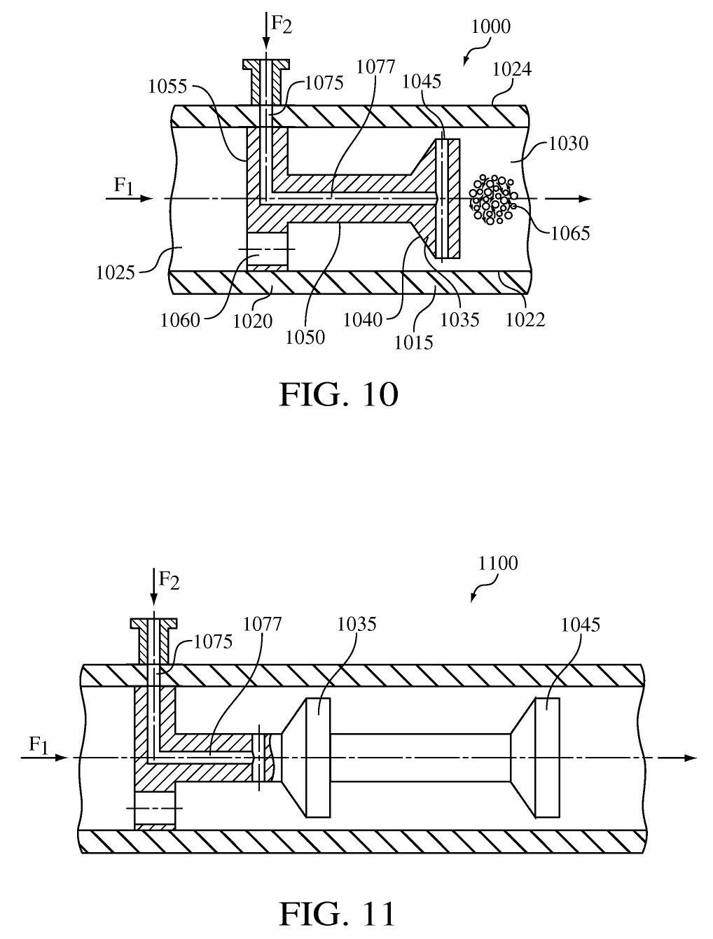

FIG. 10 illustrates another embodiment hydrodynamic cavitation crystallization device 1000 comprising a flow-through channel 1015 defined by a cylindrical wall 1020 having an inner surface 1022, an outer surface 1024, an inlet 1025 for introducing a first fluid stream F₁ (in the direction of the arrow) into the device 1000 and an outlet 1030 for exiting fluid from the device 1000.

Disposed within the flow-through channel 1015 along or near the centerline CL of the flow-through 1015 is a cavitation generator such as a baffle 1035. As shown in FIG. 10, the baffle 1035 includes a conically-shaped surface 1040 extending into a cylindrically-shaped surface 1045 wherein conically-shaped portion 1040 of the baffle 1035 confronts the fluid flow. The baffle 1035 is positioned on a stem 1050 that is connected to a disk 1055 having an orifice 60. The disk 1055 is mounted in an inlet 1025 and retains the baffle 1035 inside the flow-through channel 1015.

The baffle 1035 is configured to generate a hydrodynamic cavitation field 1065 downstream from the baffle 1035 via a the local constriction 1070 of fluid flow. In this embodiment, the local constriction 1070 is an annular orifice defined between the inner surface 1022 of the flow-through channel 1015 and the cylindrically-shaped surface 1045 of the baffle 1035.

Disposed in the cylindrical wall 1020 of the flow-through channel 1015 is a port 1075 for introducing a second fluid stream F₂ (in the direction of the arrow) into the flow-through channel 1015. Beginning at the port 1075, a fluid passage 1077 is provided that extends through the disk 1055, the stem 1050, the baffle 1035 and exits in the local constriction 1070 of flow. In a slightly different embodiment as shown in FIG. 11, a crystallization hydrodynamic cavitation device 1100 is provided, which is similar to the device 1000 illustrated in FIG. 10 in structure and operation, except that the fluid passage 1077 in the device 1100 exits upstream from the baffle 1035 and another baffle 1135 is provided downstream from the baffle 1035, thereby providing a two stage hydrodynamic cavitation process.

In operation of the device 1000 illustrated in FIG. 10, the first fluid stream F₁ enters the flow-through channel 1015 via the inlet 1025 and moves through the orifice 1060 in the direction indicated by the arrows beneath F₁. The second fluid stream F₂ enters the flow-through channel 1015 via the port 1075, flows through the fluid passage 1077, and mixes with the first fluid stream F₁ while it is passing through the local constriction 1070. In one embodiment, the first fluid stream F₁ is an anti-solvent and the second fluid stream F₂ is a feed solution. Alternatively, the first fluid stream F₁ is a feed solution and second fluid stream F₂ is an anti-solvent. In the embodiment where the first fluid stream F₁ is the anti-solvent and the second fluid stream is the feed solution, the two fluid streams can be mixed by infusing the feed solution (i.e., the second fluid stream F₂) into the anti-solvent (i.e., the first fluid stream F₁).

The mixed first and second fluid streams F₁, F₂ then pass through the local constriction 1070 of flow, where the velocity of the first and second fluid streams F₁, F₂ increases to a minimum velocity (i.e., velocity at which cavitation bubbles begin to appear) dictated by the physical properties of the first and second fluid streams F₁, F₂. Optionally, instead of a single pass of the first fluid stream F₁ through the device 1000, the first fluid stream F₁ (i.e., the anti-solvent) can be recirculated through the device 1000, while the second fluid stream F₂ (i.e., the feed solution) is being introduced to the anti-solvent via the port 1075. As the first and second fluid streams F₁, F₂ pass through the local constriction 1070 of flow, the hydrodynamic cavitation field 1065 (which generates cavitation bubbles) is formed downstream of the baffle 1035. Upon reaching an elevated static pressure zone, the bubbles collapse causing high local pressures (to 5,000 kg/cm²) and temperatures (to 15,000° C.) to effect nucleation and thereby directly produce tiny crystals. The remaining fluids exit the flow-through channel 1015 via the outlet 1030, while the product crystals isolated using conventional recovery techniques.

Typically, the first, second, and third fluid streams F₁, F₂, F₃ are fed into the devices discussed above with the aid of a pump (not shown). The type of pump selected is determined on the basis of the physiochemical properties of the pumpable medium and the hydrodynamic parameters necessary for the accomplishment of the process.

The following examples are given for the purpose of illustrating the present invention and should not be construed as limitations on the scope or spirit thereof.

### EXAMPLE 1

30 grams of technical grade NaCl (sodium chloride)-(feed solution) was dissolved into 100 ml of distilled water in a beaker. 200 ml of ethanol (antisolvent) (95% ethanol+5% methanol, Aldrick™) was added to the beaker with a volumetric ratio of anti-solvent/feeding solution=2:1.

The solution was mixed until NaCl (sodium chloride) crystals appeared. Upon completion, the product was filtered, washed, and then dried. The crystal particle size (d 90) was 150 microns.

### EXAMPLE 2

The crystallization process was carried out in a cavitation device substantially similar to the device 400 illustrated in FIG. 4 and described above. The cavitation device included a single orifice having a diameter of 0.010 inches and was capable of operating at pressures up to 8,000 psi with a nominal flow rate of up to 800 ml/min.

Ethanol (anti-solvent) was fed at 600 psi, via a high pressure pump, through the flow-through channel, while NaCl (feed solution) was introduced at 600 psi, via a high pressure pump, into flow-through channel via a port positioned upstream from the orifice at a 2:1 anti-solvent/feed solution ratio. The combined anti-solvent and feeding solution then passed through the orifice causing hydrodynamic cavitation to effect nucleation. NaCl was crystallized and discharged from cavitation device. The resultant crystal particle size (d 90) of the recovered crystalline NaCl was 30 microns.

### EXAMPLE 3

The crystallization process of Example 2 was repeated in the cavitation device 400, but at a higher hydrodynamic pressure of 3,000 psi. The resultant crystal particle size (d 90) was 20 microns.

### EXAMPLE 4

The crystallization process of Example 2 was repeated in the cavitation device 400, but at a higher hydrodynamic pressure of 6,500 psi. The resultant crystal particle size (d 90) was 14 microns.

### EXAMPLE 5

The crystallization process of Example 2 was repeated in the cavitation device 400, but at a 6:1 ratio of anti-solvent/feeding solution and at a hydrodynamic pressure of 1,000 psi. The resultant crystal particle size (d 90) was 10 microns.

### EXAMPLE 6

The crystallization process was carried out in a cavitation device substantially similar to the device 500 illustrated in FIG. 5 and described above. The cavitation device included a single orifice having a diameter of 0.010 inches.

2000 ml of ethanol (anti-solvent) was recirculated in the cavitation device 500 at 400 psi. A 250 ml solution of NaCl was introduced at 400 psi to the cavitation device 500 directly into the local constriction in orifice 450 via the entry port 455. The total time of introduction of the NaCl solution was 7 minutes. The resultant crystal particle size (d 90) was 20 microns.

### EXAMPLE 7

The crystallization process was carried out in a cavitation device substantially similar to the device 500 illustrated in FIG. 5 and described above. The cavitation device had a single orifice having a diameter of 2 mm.

Initially, 1901.2 ml of deionized water at a temperature of 18.2° C. was added to the hopper of the cavitation device. The cavitation device was then started to permit the deionized water to flow through the flow-through channel of the cavitation device, during which 1.0 g of hydroxypropyl-cellulose and 0.15 g of sodium lauryl sulfate were added to the hopper and dissolved in the deionized water to form a water phase mixture (anti-solvent, fluid stream F₁). The cavitation device was then turned off temporarily. Next, naproxen was dissolved in ethanol to prepare 105.9 ml of a 0.276% (w/w) solution (feed solution), which was kept at room temperature.

The cavitation device was then restarted with the water phase mixture already in it and the water phase mixture (fluid stream F₁) was supplied to the cavitation device at a pressure of 700 psi and at a flow rate of 12.02 liter/min. Next, the naproxen solution was placed in the dosing hopper of the cavitation device, where it was introduced into the orifice (fluid stream F₂) at a pressure of 700 psi and at a flow rate of 0.235 liter/min over a period of time equal to 2.7 passes (recirculation) of the water phase mixture through the orifice. During introduction into the orifice, the naproxen solution was kept at a temperature of 18.2° C.

At the conclusion of the process, naproxen crystals of sizes ranging from 0.13 microns to 2.44 microns were produced. The median particle size of the naproxen crystals was 0.67 microns (670 nm).

### EXAMPLE 8

The crystallization process was carried out in the same cavitation device as described in Example 7.

Initially, 1901.2 ml of deionized water at a temperature of 18.5° C. was added to the hopper of the cavitation device. The cavitation device was then started to permit the deionized water to flow through the flow-through channel of the cavitation device, during which 1.0 g of hydroxypropyl-cellulose and 0.15 g of sodium lauryl sulfate were added to the hopper and dissolved in the deionized water to form a water phase mixture (anti-solvent, fluid stream F₁). The cavitation device was then turned off temporarily. Next, naproxen was dissolved in ethanol to prepare 39.21 ml of a 0.134% (w/w) solution (feed solution), which was kept at room temperature.

The cavitation device was then restarted with the water phase mixture already in it and the water phase mixture (fluid stream F₁) was supplied to the cavitation device at a pressure of 700 psi and at a flow rate of 12.02 liter/min. Next, the naproxen solution was placed in the dosing hopper of the cavitation device, where it was introduced into the orifice (fluid stream F₂) at a pressure of 700 psi and at a flow rate of 0.235 liter/min over a period of time equal to 1.0 pass (single pass) of the water phase mixture through the orifice. During introduction into the orifice, the naproxen solution was kept at a temperature of 18.5° C.

At the conclusion of the process, naproxen crystals of sizes ranging from 0.14 microns to 3.26 microns were produced. The median particle size of the naproxen crystals was 0.92 microns (920 nm).

### EXAMPLE 9

The crystallization process was carried out in the same cavitation device as described in Example 7.

Initially, 1901.2 ml of deionized water at a temperature of 1.5° C. was added to the hopper of the cavitation device. The cavitation device was then started to permit the deionized water to flow through the flow-through channel of the cavitation device, during which 1.0 g of hydroxypropyl-cellulose and 0.15 g of sodium lauryl sulfate were added to the hopper and dissolved in the deionized water to form a water phase mixture (anti-solvent, fluid stream F₁). The cavitation device was then turned off temporarily. Next, naproxen was dissolved in ethanol to prepare 105.9 ml of a 0.276% (w/w) solution (feed solution), which was kept at room temperature.

The cavitation device was then restarted with the water phase mixture already in it and the water phase mixture (fluid stream F₁) was supplied to the cavitation device at a pressure of 100 psi and at a flow rate of 5.71 liter/min. Next, the naproxen solution was placed in the dosing hopper of the cavitation device, where it was introduced into the orifice (fluid stream F₂) at a pressure of 100 psi and at a flow rate of 0.176 liter/min over a period of time equal to 1.8 passes (recirculation) of the water phase mixture through the orifice. During introduction into the orifice, the naproxen solution was kept at a temperature of 1.5° C.

At the conclusion of the process, naproxen crystals of sizes ranging from 0.14 microns to 1.54 microns were produced. The median particle size of the naproxen crystals was 0.40 microns (400 nm).

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or multiple components.

While the present application illustrates various embodiments, and while these embodiments have been described in some detail, it is not the intention of the applicant to restrict or in any way limit the scope of the claimed invention to such detail. Additional advantages and modifications will readily appear to those skilled in the art. Therefore, the invention, in its broader aspects, is not limited to the specific details and illustrative examples shown and described. Accordingly, departures may be made from such details without departing from the spirit or scope of the applicant's claimed invention. Moreover, the foregoing embodiments are illustrative, and no single feature or element is essential to all possible combinations that may be claimed in this or a later application.

What is claimed is:

1. A process for crystallizing a compound using hydrodynamic cavitation comprising the steps of:
mixing at least one stream of a solution of such compound to be crystallized with at least one stream of an anti-solvent, wherein the compound to be crystallized is an active pharmaceutical ingredient; and
passing the mixed streams at an elevated pressure through at least one local constriction of flow to create hydrodynamic cavitation, thereby causing nucleation and the direct production of crystals of such compound.

2. The process of claim 1, wherein the mixing step occurs prior to the local constriction of flow.

3. The process of claim 1, wherein the mixing step occurs in the local constriction of flow.

4. The process of claim 1, wherein the mixing step occurs by infusing the at least one solution stream into the at least one anti-solvent stream.

5. The process of claim 4, wherein infusing the at least one solution stream into the at least one anti-solvent stream occurs during a single pass of the at least one anti-solvent stream through the at least one local constriction of flow.

6. The process of claim 4, wherein infusing the at least one solution stream into the at least one anti-solvent stream occurs during continuous recirculation of the at least one anti-solvent stream through the at least one local constriction of flow.

7. The process of claim 1, wherein the nucleation and the direct production of crystals occurs in the region of the collapsing cavitation bubbles.

8. The process of claim 1, wherein hydrodynamic cavitation is created by a cavitation generator.

9. The process of claim 1, wherein the at least one solution stream includes one or more solvents.

10. The process of claim 1, wherein one or both of, the at least one solution stream and the at least one anti-solvent stream, includes one or more surface modifiers.

11. The process of claim 11, wherein the surface modifier is selected from the group consisting of anionic surfactants, cationic surfactants, and nonionic surfactants.

12. The process of claim 11, wherein the surface modifier is a mixture of two or more surfactants.

13. The process of claim 11, wherein the surface modifier is selected from the group consisting of gelatin, casein, locithin, gum acacia, cholesterol, tragaeanth, stearic acid, benzalkonium chloride, calcium stearate, glyccryl monostearate, cetostearyl alcohol, cetomacrogol emulsifying wax, sorbitan esters, polyoxyethylcne alkyl ethers, polyoxyethylene caster oil derivatives, polyoxyethylene sorbitan htty acid esters, polyethylene glycols, polyoxyethylcne stearates, colloidel silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, and polyvinylpyrrolidone.

14. The process of claim 11, wherein the surface modifier is a phospholipid.

15. The process of claim 1, wherein the active pharmaceutical ingredient is selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products, blood substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, parathyroid biphosphonates, prostaglandins, radio-pharmaceuticals, sex hormones, anti-allergic agents, stimulants, anoretics, sympathomimetics, thyroid agents, vasodilators, and xanthines.

16. The process of claim 1, wherein the crystallized active pharmaceutical ingredient has a crystal size in the range of between about 0.01 and about 5 microns.

17. The process of claim 1, wherein the crystallized active pharmaceutical ingredient has a crystal size in the range of between about 0.01 and about 1 micron.

18. The process of claim 1, wherein the anti-solvent is capable of initiating precipitation from solution of such compound to be crystallized.

19. A method to effect nucleation in a crystallization process, the method comprising the steps of:
flowing a stream of at least one feed solution and a stream of at least one anti-solvent into a hydrodynamic cavitation crystallization device and mixing the feed solution and anti-solvent in the device to produce mixed streams, wherein the at least one feed solution includes an active pharmaceutical compound;
passing the mixed streams through a local constriction of flow in the device, thereby producing cavitation bubbles downstream from the local constriction of flow; and
collapsing the cavitation bubbles in an elevated static pressure zone, thereby temperature effecting nucleation and producing crystals.

20. The process of claim 19, wherein the active pharmaceutical compound is selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, blood products, blood substitutes, cardiac inotropic agents, contrast media, corticosteroids, cough suppressants, diagnostic agents, diagnostic imaging agents, diuretics, dopaminergics, haemostatics, immunological agents, lipid regulating agents, muscle relaxants, parasympathomimetics, parathyroid calcitonin, parathyroid biphosphonates, prostaglandins, radio-pharmaceuticals, sex hormones, anti-allergic agents, stimulants, anoretics, sympathomimetics, thyroid agents, vasodilators, and xanthines.
